(19) United States
(12) Patent Application Publication
(10) Pub. No.: US 2021/0076952 A1
(43) Pub. Date: Mar. 18, 2021

(54) COMPOUND PHYSIOLOGICAL DETECTION DEVICE

(71) Applicants: VitalSigns Technology Co., Ltd., Hsinchu County (TW); Connegence Technology Co., Ltd., Jiangsu (CN)

(72) Inventors: Jih-Liang JUANG, Hsinchu County (TW); Fung KEUNG, Jiangsu (CN)

(21) Appl. No.: 17/023,335

(22) Filed: Sep. 16, 2020

(30) Foreign Application Priority Data
Sep. 17, 2019 (CN) 201910874594.1

(51) Int. Cl. A61B 5/0205; A61B 5/0295; A61B 5/00; A61B 5/0408

(52) U.S. Cl. CPC A61B 5/02055; A61B 5/0295; A61B 5/0024; A61B 5/6801; A61B 2562/0271; A61B 5/746; A61B 5/7405; A61B 5/0006; A61B 5/0008; A61B 5/04087

(57) ABSTRACT

A compound physiological detection device is configured on a user to detect a plurality of physiological signals of the user. The compound physiological detection device comprises a body. The body comprises an electrocardiography (ECG) sensor for capturing an ECG physiological signal of the user, a photoplethysmography (PPG) sensor for capturing a blood flow change physiological signal of the user, a temperature sensor for capturing a temperature physiological signal of the user, and a sound sensor for capturing a physiological sound physiological signal of the user. The ECG sensor has a reference electrode, and the reference electrode is coupled to a temperature measuring electrode of the temperature sensor.

### CROSS-REFERENCE TO RELATED APPLICATION

This application claims priority to China Application Serial Number 201910874594.1, filed Sep. 17, 2019, which is herein incorporated by reference.

### BACKGROUND OF THE INVENTION

### Field of Invention

The disclosure relates to a physiological detection device, and more particularly, to a compound physiological detection device.

### Description of Related Art

With the continuous improvement of living standard and medical technology, people pay more and more attention to health management. Therefore, the research on various physiological monitoring and sensing technologies has gradually received attention. Therefore, whether it is a medical-grade instrument used in a hospital or a consumer-type sensing device for home health care, a variety of instruments and devices capable of measuring human physiological signals have been developed accordingly.

Especially, a kind of wearable biomedical measurement system has been gradually developed in recent years. The physiological signals of the wearer can be recorded at any time by wearing the kind of biomedical measurement system on the body. Moreover, the kind of biomedical measurement system can real-time measure the wearer's physiological signal to remind or prevent possible diseases, and can quickly remind and ask for help for the patients after surgery.

Therefore, how to obtain more accurate measurement results and how to improve prediction accuracy have become the pursued goal.

### SUMMARY

An aspect of the disclosure provides a compound physiological detection device is configured on a user to detect a plurality of physiological signals of the user. The compound physiological detection device comprises a body. The body comprises an electrocardiography (ECG) sensor for capturing an ECG physiological signal of the user, a photoplethysmography (PPG) sensor for capturing a blood flow change physiological signal of the user, a temperature sensor for capturing a temperature physiological signal of the user, and a sound sensor for capturing a physiological sound physiological signal of the user. The ECG sensor has a reference electrode, and the reference electrode is coupled to a temperature measuring electrode of the temperature sensor.

In an embodiment, the compound physiological detection device further comprises a wearing unit coupled to the body, wherein the wearing unit is attached to a skin surface of the user, and the body is attached to the wearing unit to collect the ECG physiological signal, the blood flow change physiological signal, the temperature physiological signal, and the physiological sound physiological signal.

In an embodiment, the electrocardiogram sensor has at least two conductive electrodes extending to the external surface of the body.

In an embodiment, the temperature measuring electrode of the temperature sensor extends to the external surface of the body, and the temperature measuring electrode has an outer metal shell coupled to the reference electrode.

In an embodiment, the temperature measuring electrode is disposed between the at least two conductive electrodes.

In an embodiment, the photoplethysmography (PPG) sensor has a green light source, a red light source, an infrared light source, and a light detection diode arranged in the external surface of the body.

In an embodiment, the compound physiological detection device further comprises an alarm system disposed inside the body, wherein when one of the ECG physiological signal, the blood flow change physiological signal, the temperature physiological signal, and the physiological sound physiological signal is abnormal, the alarm system issues an alarm.

In an embodiment, the compound physiological detection device further comprises a communication system for transmitting the ECG physiological signal, the blood flow change physiological signal, the temperature physiological signal, and the physiological sound physiological signal to an external device.

In an embodiment, the compound physiological detection device further comprises a barometer for measuring atmospheric pressure.

Accordingly, the compound physiological detection device can simultaneously measure the values of oxyhemoglobin saturation (SpO2), heartbeat, pulse rate, temperature, respiration rate, and electrocardiogram. Therefore, the monitor may make comprehensive judgments based on these values to accurately confirm the user's current status.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

In order to make the above and other objects, features and advantages of the disclosure more comprehensible, several embodiments accompanied with figures are described in detail below.

FIG. 1 is a schematic diagram showing the inside of a body of a compound physiological detection device according to an embodiment.

FIG. 2 is a schematic diagram showing the external surface of a body of a compound physiological detection device according to an embodiment.

### DETAILED DESCRIPTION

Reference will now be made in detail to the present preferred embodiments of the disclosure, examples of which are illustrated in the accompanying drawings. Wherever possible, the same reference numbers are used in the drawings and the description to refer to the same or like parts.

To comprehensively describe the disclosure in detail, reference may be made to the accompanying drawings and various embodiments. Meanwhile, components and steps known by the public are not described in the embodiments to prevent unnecessary limitations from being imposed to the disclosure.

Terms such as “couple” or “connect” used in the embodiments may refer to two or more components being in physical or electrical contact with each other “directly”, two or more components being in physical or electrical contact with each other “indirectly”, or acting of two or more components with each other.

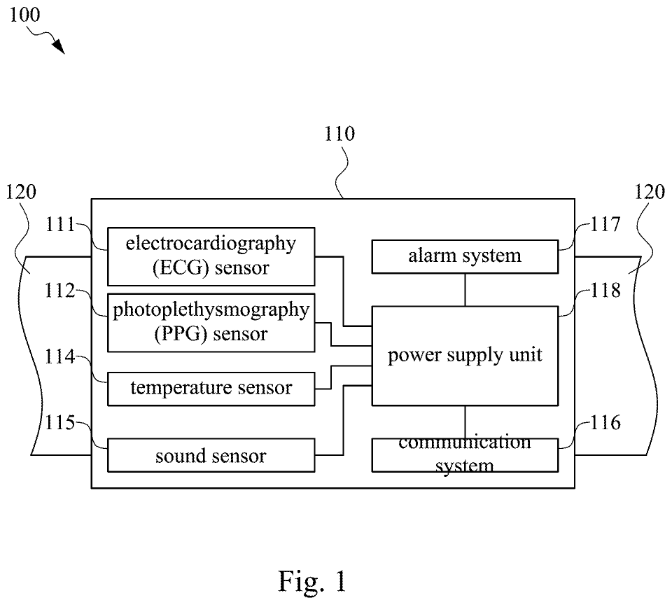

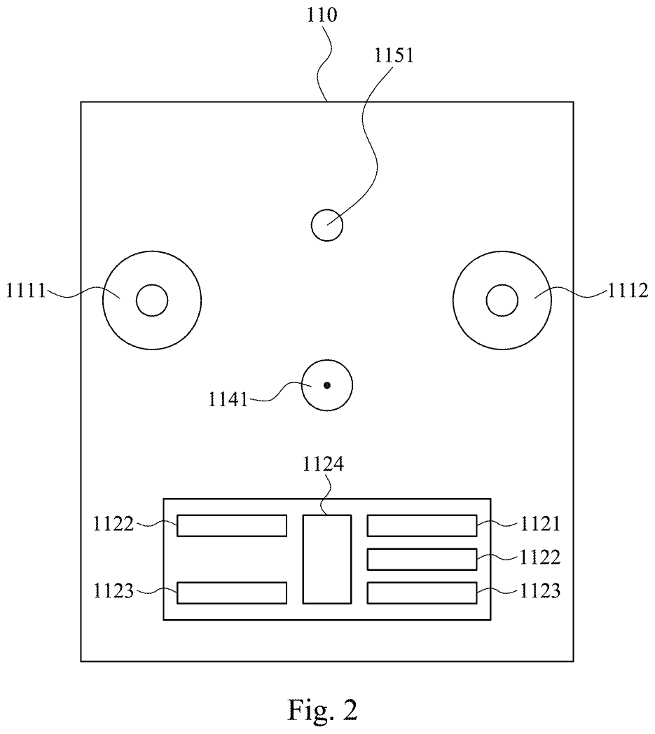

FIG. 1 is a schematic diagram showing the inside of a body of a compound physiological detection device according to an embodiment. FIG. 2 is a schematic diagram showing the external surface of a body of a compound physiological detection device according to an embodiment. Please refer to FIG. 1 and FIG. 2 at the same time. The compound physiological detection device 100 can be configured by a user to carry out real-time physiological detection. The compound physiological detection device 100 includes a body 110 and a wearing unit 120. The wearing unit 120 is configured to be attached to the skin surface of the user. The body 110 can be completely attached to the surface of the user's body through the coupling with the wearing unit 120 to collect the user's physiological signals.

The coupling between the wearing unit 120 and the body 110 may be, for example, attaching the body 110 to the wearing unit 120 in a buckle manner or touch fasteners. The wearing unit 120 has the elastic properties and the function of adjusting the length, so that the compound physiological detection device 100 can be adapted for users with different body types, or for different locations of a user.

In an embodiment, an electrocardiography (ECG) sensor 111, a photoplethysmography (PPG) sensor 112, a temperature sensor 114, and a sound sensor 115, a communication system 116, an alarm system 117, and a power supply unit 118 are disposed inside the body 110. It is worth noting that the number and type of the above sensors are only examples. In other embodiments, sensors may be added in or removed from the body 110 as needed. Moreover, at least two conductive electrodes 1111 and 1112 of the electrocardiogram sensor 111 are disposed on the external surface of the body 110. The green light source 1121, the red light source 1122, the infrared light source 1123 and the light detection diode 1124 of the photoplethysmography (PPG) sensor 112 are disposed on the external surface of the body 110. The temperature measuring electrode 1141 of the temperature sensor 114 and the sound receiving hole 1151 of the sound sensor 115 are also disposed on the external surface of the body 110. In one embodiment, the temperature measurement electrode 1141 is disposed between the two conductive electrodes 1111 and 1112.

The operation of the heart can reveal information about the human body, including health, lifestyle, and even emotional states and the early onset of heart disease. The ECG sensor 111 can detect the heartbeat rate and cardiac activity. The two conductive electrodes 1111 and 1112 are connected to the user's skin surface to measure the signal of electrical activity caused in the cardiac activity. The ECG sensor 111 receives the ECG signals captured by the two conductive electrodes 1111 and 1112 to sense a physical change between the two conductive electrodes 1111 and 1112 and the user's skin surface. Then, the ECG sensor 111 processes the ECG signals based on the physical change. The types of the two conductive electrodes 1111 and 1112 can be dry electrodes, wet gel electrodes, or other suitable ECG electrodes to adapt to different physiological conditions of different users. In addition to use the basic two conductive electrodes 1111 and 1112 for single-lead ECG measurement, in other embodiment, the present invention can also add other conductive electrodes for multi-lead ECG measurement. That is, the number of the conductive electrodes can be changed to match different ECG measurements.

Moreover, in order to provide the two conductive electrodes 1111 and 1112 a stable potential during the acquisition of the ECG signal, a reference electrode or a ground electrode is disposed to provide the stable potential. In one embodiment, the reference electrode or ground electrode is coupled to the outer metal shell of the temperature measurement electrode 1141. That is, the outer metal shell of the temperature measurement electrode 1141 is used as a reference electrode or ground electrode for providing a stable potential.

Moreover, there will be a pressure wave through the blood vessel with the heartbeat. This pressure wave will slightly change the diameter of the blood vessel. The photoplethysmography (PPG) sensor 112 is the optical technology that uses this change to obtain heart function information. The photoplethysmography (PPG) sensor 112 is used to measure oxyhemoglobin saturation (SpO2). The principle of this method is that the blood delivered in the artery changes regularly with the heart pumping cycle. The heart pumps blood (systole) and draws blood (diastole) rhythmically in a certain cycle. More blood flows through the arteries during systole, and less blood flows through the arteries during diastole. Therefore, the pulse rate can be obtained by measuring the blood flow changes in a specific part of the body. Accordingly, when the detection is performed, the lights emitted by the green light source 1121, the red light source 1122, and the infrared light source 1123 of photoplethysmography (PPG) sensor 112 penetrate into the skin, tissue and blood vessel, and then the lights are absorbed, transmitted, and reflected. Then, the change of the reflected light intensity is detected by the light detection diode 1124, and the change of blood flowing through the artery is calculated by the change. The pulse rate and the number of heartbeats can be obtained from the periodicity of the measured signal. Moreover, since the concentration change of oxyhemoglobin and deoxyhemoglobin will change the reflected light intensity, the concentration change of oxyhemoglobin and deoxyhemoglobin can be captured by detecting the reflected light intensity change. Then, the oxyhemoglobin saturation (SpO2) signal can be obtained by converting the concentration change.

The temperature sensor 114 detects the temperature of the subject through a temperature detection electrode 1141 disposed in the external surface of the body 110.

The sound sensor 115 contacts the skin of the user through the sound receiving hole 1151 disposed in the external surface of the body 110 to collect the physiological sound of the user. The physiological sound is converted into a digital signal by an analog-to-digital converter (not shown). Accordingly, the electrocardiogram sensor 111 and the sound sensor 115 can be used to simultaneously collect the electrocardiogram signal and the physiological sound of the heart or lungs, so that medical personnel can obtain medical data, such as heart sounds, lung sounds and electrocardiogram, in real time.

The communication system 116 can communicate with an external device (not shown in the figure), such as a remote server. In an embodiment, the communication system 116 can communicate with the remote server in a wired or wireless manner. Accordingly, the compound physiological detection device 100 can transmit the detection result to the remote server to store and diagnose, and receive the signal transmitting from the remote server through the communication system 116 to communicate with the remote personnel. In one embodiment, when the compound physiological detection device 100 detects that the user's physiological information is abnormal, the communication system 116 will send a signal to the remote server for subsequent processing, such as to notify relevant persons, to achieve the purpose of remote monitoring.

The alarm system 117 includes a speaker. Accordingly, when the compound physiological detection device 100 detects an abnormal physiological information of the user, an alarm is issued to notify the personnel to pay attention to the user. Therefore, the abnormal physiological information can be treated in real-time. In one embodiment, the alarm system 117 can be activated simultaneously with the communication system 116. When the physiological information is abnormal, the communication system 116 will send a signal to the remote server for subsequent processing, and the alarm system 117 sends a warning signal to inform the personnel.

The power supply unit 118 provides power to the electrocardiogram sensor 111, the photoplethysmography (PPG) sensor 112, the temperature sensor 114, the sound sensor 115, the communication system 116, and the alarm system 117. The power supply unit 118 may be, but not limited to, an alkaline battery, a rechargeable battery, or a thin mercury battery.

Furthermore, in other embodiments, because many cardiac emergencies are related to the altitude at that time, a barometer may be provided in the present invention to measure atmospheric pressure.

Accordingly, the compound physiological detection device can simultaneously measure the values of oxyhemoglobin saturation (SpO2), heartbeat, pulse rate, temperature, respiration rate, and electrocardiogram. Therefore, the monitor may make comprehensive judgments based on these values to accurately confirm the user's current status. In addition, the compound physiological detection device further includes a communication system and an alarm system. These values can be transmitted to a remote server for remote monitoring through the communication system. The compound physiological detection device can also issue a warning signal to notify the relevant personnel in real-time when abnormal values are found through the alarm system.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims and their equivalents.

What is claimed is:

1. A compound physiological detection device configured on a user to detect a plurality of physiological signals of the user, comprising:
a body, comprising:
an electrocardiography (ECG) sensor for capturing an ECG physiological signal of the user;
a photoplethysmography (PPG) sensor for capturing a blood flow change physiological signal of the user;
a temperature sensor for capturing a temperature physiological signal of the user; and
a sound sensor for capturing a physiological sound physiological signal of the user, wherein the ECG sensor has a reference electrode, and the reference electrode is coupled to a temperature measuring electrode of the temperature sensor.

2. The compound physiological detection device as claimed in claim 1, further comprising a wearing unit coupled to the body, wherein the wearing unit is attached to a skin surface of the user, and the body is attached to the wearing unit to collect the ECG physiological signal, the blood flow change physiological signal, the temperature physiological signal, and the physiological sound physiological signal.

3. The compound physiological detection device as claimed in claim 1, wherein the electrocardiogram sensor has at least two conductive electrodes extending to an external surface of the body.

4. The compound physiological detection device as claimed in claim 1, wherein the temperature measuring electrode of the temperature sensor extends to an external surface of the body, and the temperature measuring electrode has an outer metal shell coupled to the reference electrode.

5. The compound physiological detection device as claimed in claim 4, wherein the temperature measuring electrode is disposed between the at least two conductive electrodes.

6. The compound physiological detection device as claimed in claim 1, wherein the photoplethysmography (PPG) sensor has a green light source, a red light source, an infrared light source, and a light detection diode arranged in an external surface of the body.

7. The compound physiological detection device as claimed in claim 1, further comprising an alarm system disposed inside the body, wherein when one of the ECG physiological signal, the blood flow change physiological signal, the temperature physiological signal, and the physiological sound physiological signal is abnormal, the alarm system issues an alarm.

8. The compound physiological detection device of claim 1, further comprising a communication system for transmitting the ECG physiological signal, the blood flow change physiological signal, the temperature physiological signal, and the physiological sound physiological signal to an external device.

9. The compound physiological detection device of claim 1, further comprising a barometer for measuring atmospheric pressure.
